(19) United States
(12) Patent Application Publication
(10) Pub. No.: US 2008/0230061 A1
(43) Pub. Date: Sep. 25, 2008

(54) SETTING EXPIRATORY TIME IN MANDATORY MECHANICAL VENTILATION BASED ON A DEVIATION FROM A STABLE CONDITION OF END TIDAL GAS CONCENTRATIONS

(75) Inventor: Robert Quinyew Tham, Middleton, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/690,618

(22) Filed: Mar. 23, 2007

(51) Int. Cl. A61M 16/00

(52) U.S. Cl. 128/204.21

(57) ABSTRACT

A method of setting expiratory time in controlled mechanical ventilation varies a subject's expiratory times; determines end tidal gas concentrations associated with the expiratory times; establishes a stable condition of the gas concentrations; and determines an optimal expiratory time based on a deviation from the stable condition. A device for use in controlled mechanical ventilation comprises means for the same.

### FIELD OF INVENTION

In general, the inventive arrangements relate to respiratory care, and more specifically, to improvements in controlling mandatory mechanical ventilation.

### BACKGROUND OF INVENTION

Referring generally, when patients are medically unable to breathe on their own, mechanical, or forced, ventilators can sustain life by providing requisite pulmonary gas exchanges on behalf of the patients. Accordingly, modern ventilators usually include electronic and pneumatic control systems that control the pressure, flow rates, and/or volume of gases delivered to, and extracted from, patients needing medical respiratory assistance. Oftentimes, such control systems include a variety of knobs, dials, switches, and the like, for interfacing with treating clinicians, who support the patient's breathing by adjusting the afore-mentioned pressure, flow rates, and/or volume of the patient's pulmonary gas exchanges, particularly as the condition and/or status of the patient changes. Even today, however, such parameter adjustments, although highly desirable, remain challenging to control accurately, particularly using present-day arrangements and practices.

Referring now more specifically, ventilation is a complex process of delivering oxygen to, and removing carbon dioxide from, alveoli within patients' lungs. Thus, whenever a patient is ventilated, that patient becomes part of a complex, interactive system that is expected to promote adequate ventilation and gas exchange on behalf of the patient, eventually leading to the patient's stabilization, recovery, and ultimate ability to return to breathing normally and independently.

Not surprisingly, a wide variety of mechanical ventilators are available today. Most allow their operating clinicians to select and use several modes of ventilation, either individually and/or in various combinations, using various ventilator setting controls.

These mechanical ventilation modes are generally classified into one (1) of two (2) broad categories: a) patient-triggered ventilation, and b) machine-triggered ventilation, the latter of which is also commonly referred to as controlled mechanical ventilation (CMV). In patient-triggered ventilation, the patient determines some or all of the timing of the ventilation parameters, while in CMV, the operating clinician determines all of the timing of the ventilation parameters. Notably, the inventive arrangements described hereinout will be particularly relevant to CMV.

In recent years, mechanical ventilators have become increasingly sophisticated and complex, due, in large part, to recently-enhanced understandings of lung pathophysiology. Technology also continues to play a vital role. For example, many modern ventilators are now microprocessor-based and equipped with sensors that monitor patient pressure, flow rates, and/or volumes of gases, and then drive automated responses in response thereto. As a result, the ability to accurately sense and transduce, combined with computer technology, makes the interaction between clinicians, ventilators, and patients more effective than ever before.

Unfortunately, however, as ventilators become more complicated and offer more options, the number and risk of potentially dangerous clinical decisions increases as well. Thus, clinicians are often faced with expensive, sophisticated machines, yet few follow clear, concise, and/or consistent guidelines for maximal use thereof. As a result, setting, monitoring, and interpreting ventilator parameters can devolve into empirical judgment, leading to less than optimal treatment, even by well-intended practitioners.

Complicating matters ever further, ventilator support should be individually tailored for each patient's existing pathophysiology, rather than deploying a generalized approach for all patients with potentially disparate ventilation needs.

Pragmatically, the overall effectiveness of assisted ventilation will continue to ultimately depend on mechanical, technical, and physiological factors, with the clinician-ventilator-patient interface invariably continuing to play a key role. Accordingly, technology that demystifies these complex interactions and provides appropriate information to effectively ventilate patients is needed.

In accordance with the foregoing, it remains desirable to provide maximally effective mechanical ventilation parameters, particularly engaging clinicians to supply appropriate quantities and qualities of ventilator support to patients, customized for each individual patient's particular ventilated pathophysiology.

### SUMMARY OF INVENTION

In one embodiment, a method of setting expiratory time in controlled mechanical ventilation varies a subject's expiratory times; determines end tidal gas concentrations associated with the expiratory times; establishes a stable condition of the gas concentrations; and determines an optimal expiratory time based on a deviation from the stable condition.

In another embodiment, a device for use in controlled mechanical ventilation comprises means for the same.

### BRIEF DESCRIPTION OF SEVERAL VIEWS OF THE DRAWINGS

A clear conception of the advantages and features constituting inventive arrangements, and of various construction and operational aspects of typical mechanisms provided by such arrangements, are readily apparent by referring to the following illustrative, exemplary, representative, and/or non-limiting figures, which form an integral part of this specification, in which like numerals generally designate the same elements in the several views, and in which:

FIG. 1 depicts a front perspective view of a medical system comprising a ventilator;

FIG. 2 depicts a block diagram of a medical system providing ventilator support to a patient;

FIG. 3 depicts a block diagram of a ventilator providing ventilator support to the patient;

FIG. 4 depicts a flow diagram of the patient's inspiratory time (T_{I}), expiratory time (T_{E}), and forced inhalation time (T_{INH}) for a single breath, particularly during pressure controlled mechanical ventilation (CMV);

FIG. 5 depicts a flowchart of a simplified arrangement for setting the patient's inspiratory time (T_{I}) based on the patient's forced inhalation time (T_{INH});

FIG. 6 depicts a flowchart of a simplified arrangement for setting the patient's inspiratory time (T_{I}) based on when the patient's forced inhalation flow ceases;

FIG. 7 depicts a flowchart of a simplified arrangement for setting the patient's inspiratory time (T_{I}) based on when the patient's tidal volume is inspired;

FIG. 8 depicts a response curve of the patient's delivered expiratory time (dT_{E}) and exhaled CO₂ levels (F_{ET}CO₂);

FIG. 9 depicts the delivered expiratory time (dT_{E}) response curve of FIG. 8, graphically depicting an arrangement to identify the patient's optimal expiratory time (T_{E-OPTIMAL}); and

FIG. 10 depicts a response curve of the patient's delivered expiratory time (dT_{E}) and exhaled VCO₂ levels.

### DETAILED DESCRIPTION OF VARIOUS PREFERRED EMBODIMENTS

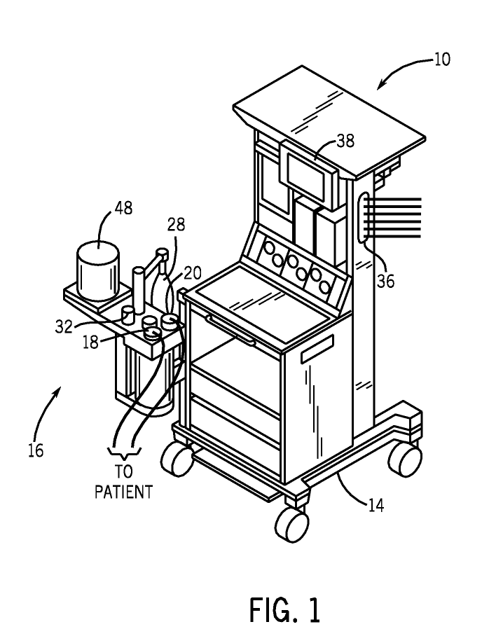

Referring now to the figures, and in particular to FIGS. 1-3, a medical system 10 is depicted for mechanically ventilating a patient 12. More specifically, an anesthesia machine 14 includes a ventilator 16, the latter having suitable connectors 18, 20 for connecting to an inspiratory branch 22 and expiratory branch 24 of a breathing circuit 26 leading to the patient 12. As will be subsequently elaborated upon, the ventilator 16 and breathing circuit 26 cooperate to provide breathing gases to the patient 12 via the inspiratory branch 22 and to receive gases expired by the patient 12 via the expiratory branch 24.

If desired, the ventilator 16 can also be provided with a bag 28 for manually bagging the patient 12. More specifically, the bag 28 can be filled with breathing gases and manually squeezed by a clinician (not shown) to provide appropriate breathing gases to the patient 12. Using this bag 28, or “bagging the patient,” is often required and/or preferred by the clinicians, as it can enable them to manually and/or immediately control delivery of the breathing gases to the patient 12. Equally important, the clinician can sense conditions in the respiration and/or lungs 30 of the patient 12 according to the feel of the bag 28, and then accommodate for the same. While it can be difficult to accurately obtain this feedback while mechanically ventilating the patient 12 using the ventilator 16, it can also fatigue the clinician if the clinician is forced to bag the patient 12 for too long a period of time. Thus, the ventilator 16 can also provide a toggle 32 for switching and/or alternating between manual and automated ventilation.

In any event, the ventilator 16 can also receive inputs from sensors 34 associated with the patient 12 and/or ventilator 16 at a processing terminal 36 for subsequent processing thereof, and which can be displayed on a monitor 38, which can be provided by the medical system 10 and/or the like. Representative data received from the sensors 34 can include, for example, inspiratory time (T_{I}), expiratory time (T_{E}), forced inhalation time (T_{INH}), respiratory rates (f), I:E ratios, positive end expiratory pressure (PEEP), fractional inspired oxygen (F_{I}O₂), fractional expired oxygen (F_{E}O₂), breathing gas flow (F), tidal volumes (V_{T}), temperatures (T), airway pressures (P_{aw}), arterial blood oxygen saturation levels (SₐO₂), blood pressure information (BP), pulse rates (PR), pulse oximetry levels (SₚO₂), exhaled CO₂ levels (F_{ET}CO₂), concentration of inspired inhalation anesthetic agent (C_{I} agent), concentration of expired inhalation anesthetic agent (C_{E} agent), arterial blood oxygen partial pressure (PₐO₂), arterial carbon dioxide partial pressure (PₐCO₂), and the like.

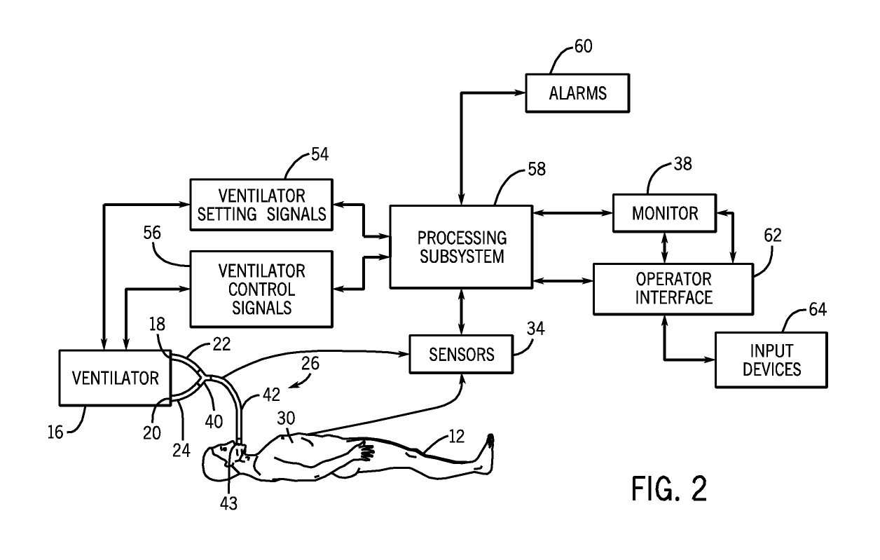

Referring now more specifically to FIG. 2, the ventilator 16 provides breathing gases to the patient 12 via the breathing circuit 26. Accordingly, the breathing circuit 26 typically includes the afore-mentioned inspiratory branch 22 and expiratory branch 24. Commonly, one end of each of the inspiratory branch 22 and expiratory branch 24 is connected to the ventilator 16, while the other ends thereof are usually connected to a Y-connector 40, which can then connect to the patient 12 through a patient branch 42, which can also include an interface 43 to secure the patient's 12 airways to the breathing circuit 26 and/or prevent gas leakage out thereof.

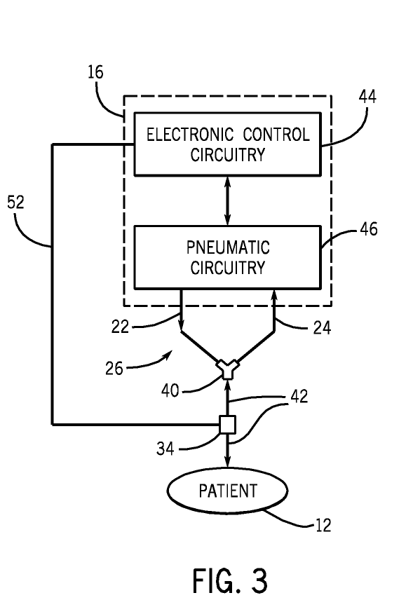

Referring now more specifically to FIG. 3, the ventilator 16 can also include electronic control circuitry 44 and/or pneumatic circuitry 46. More specifically, various pneumatic elements of the pneumatic circuitry 46 provide breathing gases to the lungs 30 of the patient 12 through the inspiratory branch 22 of the breathing circuit 26 during inhalation. Upon exhalation, the breathing gases are discharged from the lungs 30 of the patient 12 and into the expiratory branch 24 of the breathing circuit 26. This process can be iteratively enabled by the electronic control circuitry 44 and/or pneumatic circuitry 46 in the ventilator 16, which can establish various control parameters, such as the number of breaths per minute to administer to the patient 12, tidal volumes (V_{T}), maximum pressures, etc., that can characterize the mechanical ventilation that the ventilator 16 supplies to the patient 12. As such, the ventilator 16 may be microprocessor based and operable in conjunction with a suitable memory to control the pulmonary gas exchanges in the breathing circuit 26 connected to, and between, the patient 12 and ventilator 16.

Even more specifically, the various pneumatic elements of the pneumatic circuitry 46 usually comprise a source of pressurized gas (not shown), which can operate through a gas concentration subsystem (not shown) to provide the breathing gases to the lungs 30 of the patient 12. This pneumatic circuitry 46 may provide the breathing gases directly to the lungs 30 of the patient 12, as typical in a chronic and/or critical care application, or it may provide a driving gas to compress a bellows 48 (see FIG. 1) containing the breathing gases, which can, in turn, supply the breathing gases to the lungs 30 of the patient 12, as typical in an anesthesia application. In either event, the breathing gases iteratively pass from the inspiratory branch 22 to the Y-connector 40 and to the patient 12, and then back to the ventilator 16 via the Y-connector 40 and expiratory branch 24.

In the embodiment depicted in FIG. 3, one or more of the sensors 34, placed in the breathing circuit 26, can also provide feedback signals back to the electronic control circuitry 44 of the ventilator 16, particularly via a feedback loop 52. More specifically, a signal in the feedback loop 52 could be proportional, for example, to gas flows and/or airway pressures in the patient branch 42 leading to the lungs 30 of the patient 12. Inhaled and exhaled gas concentrations (such as, for example, oxygen O₂, carbon dioxide CO₂, nitrous oxide N₂O, and inhalation anesthetic agents), flow rates (including, for example, spirometry), and gas pressurization levels, etc., are also representative feedback signals that could be captured by the sensors 34, as can the time periods between when the ventilator 16 permits the patient 12 to inhale and exhale, as well as when the patient's 12 natural inspiratory and expiratory flows cease.

Accordingly, the electronic control circuitry 44 of the ventilator 16 can also control displaying numerical and/or graphical information from the breathing circuit 26 on the monitor 38 of the medical system 10 (see FIG. 1), as well as other patient 12 and/or system 10 parameters from other sensors 34 and/or the processing terminal 36 (see FIG. 1). In other embodiments, various components of which can also be integrated and/or separated, as needed and/or desired.

By techniques known in the art, the electronic control circuitry 44 can also coordinate and/or control, among other things, for example, other ventilator setting signals 54, ventilator control signals 56, and/or a processing subsystem 58, such as for receiving and processing signals, such as from the sensors 34, display signals for the monitor 38 and/or the like, alarms 60, and/or an operator interface 62, which can include one or more input devices 64, etc., all as needed and/or desired and interconnected appropriately (e.g., see FIG. 2). These components are functionally depicted for clarity, wherein various ones thereof can also be integrated and/or separated, as needed and/or desired. For further enhanced clarity, other functional components should also be well-understood but are not shown—e.g., one or more power supplies for the medical system 10 and/or anesthesia machine 14 and/or ventilator 16, etc. (not shown).

Now then, against this background, the inventive arrangements establish ventilation parameters according to patient physiology. These arrangements, to be now described, allow clinicians to control patient ventilation parameters throughout the patient's 12 respiratory cycle and enables ventilation treatments to be individually optimized for patients 12 subject to pressure controlled mechanical ventilation (CMV).

Referring generally, pressure controlled mechanical ventilation (CMV) consists of a decelerating inspiratory gas flow, for example as resulting from a pressure controlled ventilation (PCV) mode whereby flow ceases when the patient's 12 inflated lung pressure equilibrates with the inspired pressure (P_{INSP}), which can be a user settable parameter in PCV ventilation mode. Such a decelerating flow pattern can also be experienced when a ventilator 16 delivers a predetermined short volume pulse into a breathing circuit 26 and allows the gas pressure in the breathing circuit 26 to equilibrate within the patient's 12 lungs 30. When pressure equilibration occurs between the breathing circuit 26 and the patient's 12 lungs 30, inspiratory flow ceases. One can also appreciate that during the inspiratory phase of ventilation, there are other ventilator flow patterns that can rapidly force an anticipated gas volume by initially delivering a high ventilator flow followed by a flow reduction to zero or nearly zero flow. In response to this forced inhalation, gas flow to the patient's 12 lungs 30 decelerates to zero or near zero when the desired tidal volume (V_{T}) is attained. Hereinout, these ventilator control methods are included as representative pressure controlled ventilation (PCV). In particular, pressure controlled ventilation (PCV) has delivers the tidal gas volume V_{T} to the patient 12 over a generally shorter time than a constant flow volume control ventilation (VCV) mode. In VCV, for example, the ventilator 16 delivers a constant flow over the entire set inspiratory times (sT_{I}). The early delivery of the entire tidal gas volume V_{T} in PCV verses VCV allows more gases in the patient's 12 lungs 30 to exchange with the patient's 12 pulmonary blood early in the inspiratory phase of ventilation, making PCV generally more efficient in removing or adding gases into the patient's 12 blood than VCV. This is particularly evident for a patient 12 who is being ventilated at high respiration rate or for gases that diffuse more slowly through the patient's 12 alveolar to the patient's 12 blood.

To facilitate the following discussion, the following generalized and/or representative explanations and/or definitions may be referred to:

1. T_{I} is Inspiratory Time.

More specifically, T_{I} is the amount of time, measured in seconds, set on the ventilator 16 by the clinician, lasting from the beginning of the patient's 12 inspiration to the beginning of the patient's 12 expiration. Accordingly, T_{I} is the patient's 12 inspiratory time.

Inspiratory times T_{I} can be further broken down into a set inspiratory time sT_{I}, a delivered inspiratory time dT_{I}, and a measured inspiratory time mT_{I}. More specifically, the set inspiratory time sT_{I} is the amount of time that the clinician sets on the ventilator 16 to deliver gases to the patient 12 during inspiration, while the delivered inspiratory time dT_{I} is the amount of time that gases are actually allowed to be delivered to the patient 12 from the ventilator 16 during inspiration. Similarly, the measured inspiratory time mT_{I} is the amount of time that the ventilator 16 measures for allowing gases to be delivered to the patient 12 during inspiration. Ideally, the set inspiratory time sT_{I}, delivered inspiratory time dT_{I}, and measured inspiratory time mT_{I} are equal or substantially equal. However, if the clinician or ventilator 16 is searching for an optimal inspiratory time T_{I}, as elaborated upon below, then each of these inspiratory times T_{I} may be different or slightly different. For example, the clinician and/or ventilator 16 may have established a set inspiratory time sT_{I}, yet the delivered inspiratory time dT_{I} may deviate therefrom in the process of searching for, for example, the patient's 12 forced inhalation time T_{INH}.

2. T_{E} is Expiratory Time.

More specifically, T_{E} is the amount of time, measured in seconds, set on the ventilator 16 by the clinician, lasting from the beginning of the patient's 12 expiration to the beginning of the patient's 12 inspiration. Accordingly, T_{E} is the patient's 12 expiratory time.

Like inspiratory times T_{I}, expiratory times T_{E} can also be further broken down into a set expiratory time sT_{E}, a delivered expiratory time dT_{E}, and a measured expiratory time mT_{E}. More specifically, the set expiratory time sT_{E} is the amount of time that the clinician sets on the ventilator 16 to allow the patient 12 to exhale gases during expiration, while the delivered expiratory time dT_{E} is the amount of time that gases are allowed to be exhaled by the patient 12 during expiration. Similarly, the measured expiratory time mT_{E} is the amount of time that the ventilator 16 measures for having allowed the patient 12 to exhale gases during expiration. Ideally, the set expiratory time sT_{E}, delivered expiratory time dT_{E}, and measured expiratory time mT_{E} are equal or substantially equal. However, if the clinician or ventilator 16 is searching for an optimal expiratory time T_{E-OPTIMAL}, as elaborated upon below, then each of these expiratory times T_{E} may be different or slightly different. For example, the clinician and/or ventilator 16 may have established a set expiratory time sT_{E}, yet the delivered expiratory time dT_{E} may deviate therefrom in the process of searching, for example, for the patient's 12 optimal expiratory time T_{E-OPTIMAL}.

3. I:E Ratios are Ratios Between T_{I} and T_{E}.

More specifically, I:E ratios measure inspiratory times divided by expiratory times—i.e., T_{I}/T_{E}, which is commonly expressed as a ratio. Common I:E ratios are 1:2, meaning patients 12 may inhale for a certain period of time (x) and then exhale for twice as long (2×). However, since some patients 12 may have obstructed pathologies (e.g., chronic obstructive pulmonary disease (COPD)) and/or slower exhalation, requiring the clinician to set longer expiratory times T_{E}, I:E ratios can also be set at ratios closer to 1:3 and/or 1:4, particularly to provide the necessary expiratory time T_{E} for a given patient 12 to fully exhale, although I:E ratios from 1:8 and 2:1 are also not uncommon, with common ventilators 16 providing 0.5 gradations therebetween.

4. T_{INH} is Forced Inhalation Time.

More specifically, T_{INH} is the amount of time, measured in seconds, required for the patient's 12 forced inhalation flow to cease during pressure controlled mechanical ventilation. Accordingly, T_{INH} is the patient's 12 forced inhalation time.

Oftentimes in pressure controlled mechanical ventilation, the patient's 12 inspiratory time T_{I} does not equal the patient's 12 forced inhalation time T_{INH}—i.e., the patient's 12 inspiratory time T_{I}, as set by the clinician on the ventilator 16, often does not coincide with the patient's 12 forced inhalation time T_{INH}. Moreover, in accordance with many default settings on many ventilators 16, respiratory rates f (see below) are commonly set between 6-10 breaths/minute and I:E ratios are commonly set at 1:2, resulting in many clinicians setting inspiratory times T_{I} between 2.0-3.3 seconds, as opposed to typical inhalation times T_{INH} being less than or equal to approximately 0.8-1.5 seconds. Several of the inventive arrangements, on the other hand, set the patient's 12 inspiratory times Ti approximately equal to the patient's 12 forced inhalation times T_{INH} (i.e., 2*T_{INH}≧T_{I}≧T_{INH}).

If the clinician or ventilator 16 sets the patient's 12 inspiratory time T_{I} less than or equal to the patient's 12 forced inhalation time T_{INH}, there can be inadequate time for the patient 12 to inspire the gases in the patient's 12 lungs 30. This can result in insufficient breath volume in the patient's 12 lungs 30, thereby inadvertently and/or unknowingly under-ventilating the patient's 12 lungs 30. Accordingly, several of the inventive arrangements set the patient's 12 inspiratory time T_{I} approximately equal to the patient's 12 forced inhalation time T_{INH}, preferably with the patient's 12 inspiratory time T_{I} being set greater than or equal to the patient's 12 force inhalation time T_{INH}.

5. PEEP is Positive End Expiratory Pressure.

More specifically, PEEP is the patient's 12 positive end expiratory pressure, often measured in cmH₂0. Accordingly, PEEP is the amount of pressure in the patient's 12 lungs 30 at the end of the patient's 12 expiratory time T_{E}, as controlled by the ventilator 16.

Like inspiratory times T_{I} and expiratory times T_{E}, positive end expiratory pressure PEEP can also be further broken down into a set positive end expiratory pressure sPEEP, a measured positive end expiratory pressure mPEEP, and a delivered positive end expiratory pressure dPEEP. More specifically, the set positive end expiratory pressure sPEEP is the amount of pressure that the clinician sets on the ventilator 16 for the patient 12, while the measured positive end expiratory pressure mPEEP is the amount of pressure in the patient's 12 lungs 30 at the end of the patient's 12 expiratory time T_{E}. Similarly, the delivered positive end expiratory pressure dPEEP is the amount of pressure delivered by the ventilator to the patient 12. Usually, the set positive end expiratory pressure sPEEP, measured positive end expiratory pressure mPEEP, and delivered positive end expiratory pressure dPEEP are equal or substantially equal. However, the measured positive end expiratory pressure mPEEP can be greater than the set positive end expiratory pressure sPEEP when breath stacking, for example, occurs.

6. F_{I}0₂ is Fraction of Inspired Oxygen.

More specifically, F_{I}0₂ is the concentration of oxygen in the patient's 12 inspiratory gas, often expressed as a fraction or percentage. Accordingly, F_{I}0₂ is the patient's 12 fraction of inspired oxygen.

7. F_{E}0₂ is Fraction of Expired Oxygen.

More specifically, F_{E}0₂ is the concentration of oxygen in the patient's 12 expiratory gas, often expressed as a fraction or percentage. Accordingly, F_{E}0₂ is the patient's 12 fraction of expired oxygen.

8. f is Respiratory Rate.

More specifically, f is the patient's 12 respiratory rate, measured in breaths/minute, set on the ventilator 16 by the clinician.

9. V_{T} is Tidal Volume.

More specifically, V_{T} is the total volume of gases, measured in milliliters, delivered to the patient's 12 lungs 30 during inspiration. Accordingly, V_{T} is the patient's 12 tidal volume.

Like inspiratory times T_{I} and expiratory times T_{E}, tidal volumes V_{T} can also be further broken down into a set tidal volume sV_{T}, a delivered tidal volume dV_{T}, and a measured tidal volume mV_{T}. More specifically, the set tidal volume sV_{T} is the volume of gases that the clinician sets on the ventilator 16 to deliver gases to the patient 12 during inspiration, while the delivered tidal volume dV_{T} is the volume of gases actually delivered to the patient 12 from the ventilator 16 during inspiration. Similarly, the measured tidal volume mV_{T} is the volume of gases that the ventilator 16 measures for having delivered gases to the patient 12 during inspiration. Ideally, the set tidal volume sV_{T}, delivered tidal volume dV_{T}, and measured tidal volume mV_{T} are equal or substantially equal. However, if the clinician or ventilator 16 is searching for a set optimal tidal volume sV_{T}, as elaborated upon below, then each of these set tidal volumes sV_{T} may be different or slightly different.

10. F_{ET}CO₂ is end Tidal Carbon Dioxide CO₂.

More specifically, F_{ET}CO₂ is the concentration of carbon dioxide CO₂ in the patient's 12 exhaled gas, often expressed as a fraction or percentage. Accordingly, F_{ET}CO₂ is the amount of carbon dioxide CO₂ exhaled by the patient 12 at the end of a given breath.

11. VCO₂ is the Volume of Carbon Dioxide CO₂ per Breath.

More specifically, VCO₂ is the volume of carbon dioxide CO₂ that the patient 12 exhales in a single breath. Accordingly, VCO₂ is the patient's 12 volume of CO₂ exhaled per breath.

Now then, clinicians usually begin ventilation by selecting an initial set tidal volume sV_{T}, respiratory rate f, and I:E ratio. The respiratory rate f and I:E ratio usually determine the initial set inspiratory time sT_{I} and initial set expiratory time sT_{E} that the clinician sets on the ventilator 16. In other words, the actual set inspiratory time sT_{I} and actual set expiratory time sT_{E} that the clinician uses are usually determined in accordance with the following equations:

$$f = \frac{60}{{sT}_{I} + {sT}_{E}}$$

$$I \text{:} E = \frac{{sT}_{I}}{{sT}_{E}}$$

Moreover, the clinician usually makes these initial determinations based on generic rule-of-thumb settings, taking into account factors such as, for example, the patient's 12 age, weight, height, gender, geographical location, etc. Once the clinician makes these initial determinations, the inventive arrangements can now be appreciated.

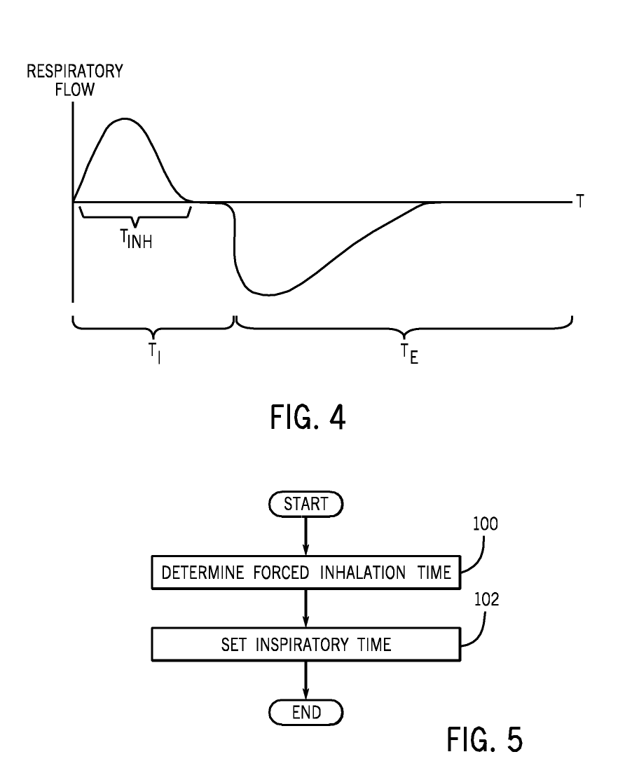

Referring now to FIG. 4, a graph of the relation between delivered inspiratory time dT_{I}, delivered expiratory time dT_{E}, and forced inhalation time T_{INH} is depicted for a single breathing cycle for a patient 12 undergoing pressure controlled mechanical ventilation (CMV). As can be seen in the figure, the patient's 12 delivered inspiratory time dT_{I} is greater than the patient's 12 forced inhalation time T_{INH}, as can be viewed by the measured inspiratory time mT_{I}.

Referring now to FIG. 5, a flowchart depicts a simplified arrangement for setting the patient's 12 set inspiratory time sT_{I} based on the patient's 12 forced inhalation time T_{INH}. More specifically, a method begins in a step 100, during which the patient's 12 forced inhalation time T_{INH} is determined. Preferably, the patient's 12 forced inhalation time T_{INH} is determined using the patient's 12 airway flow waveform, particularly when the first derivative thereof approaches zero, as is well-known in the art. Alternatively, other arrangements are also well-known in the art and can also be used to determine the patient's 12 forced inhalation time T_{INH} in step 100, such as, for example, airway flow analysis of the patient 12; tidal volume V_{T} analysis of the patient 12; acoustic analysis of the patient 12; vibration analysis of the patient 12; airway pressure analysis P_{aw} of the patient 12; capnographic morphology analysis of the patient 12; respiratory mechanics analysis of the patient 12; and/or thoracic excursion corresponding to gases exhaled from the lungs 30 of the patient 12 (e.g., imaging the patient 12, plethysmographic analysis of the patient 12, and/or electrical impedance tomography analysis of the patient, and/or the like), etc.

Thereafter, the patient's 12 forced inhalation time T_{INH} can be used to set the patient's 12 set inspiratory time sT_{I} on the ventilator 16. More specifically, the patient's 12 set inspiratory time sT_{I} can be set based on the patient's 12 forced inhalation time T_{INH}, and, for example, set equal or substantively equal to the patient's 12 forced inhalation time T_{INH}, as shown in a step 102 in FIG. 5, after which the method ends.

Now then, in accordance with the foregoing, the patient's 12 set inspiratory time sT_{I} is preferably set equal to, or slightly greater than, the patient's 12 forced inhalation time T_{INH}.

If, however, the patient's 12 forced inhalation flow does not cease, or effectively decrease to an insignificant level so as not to add substantive gas volume to the tidal volume V_{T}, at the end of the patient's 12 ventilated set inspiratory time sT_{I}, as set by the clinician and/or ventilator, then the clinician can increase the patient's 12 set inspiratory time sT_{I} until the patient's 12 forced inhalation flow ceases, or effectively decreases to an insignificant level.

As previously noted, the patient's 12 spontaneous breathing is controlled by numerous reflexes that control the patient's 12 respiratory rates f and tidal volumes V_{T}. Particularly during pressure controlled mechanical ventilation (CMV), however, these reflexes are either obtunded and/or overwhelmed. In fact, one of the only aspects of ventilation that usually remains under the patient's 12 control is the patient's 12 forced inhalation time T_{INH}, as required for a given volume, as previously elaborated upon. This is why it can be used to set the patient's 12 set inspiratory time sT_{I} on the ventilator 16 based thereon.

Now then, the inventive arrangements utilize the patient's 12 forced inhalation time T_{INH} and/or physiological parameters to determine and/or set the patient's 12 set inspiratory time sT_{I}, set expiratory time sT_{E}, and/or set tidal volume sV_{T}, either directly and/or indirectly. For example, the patient's 12 expiratory time T_{E} may be set directly, or may it be determined by the respiratory rate f for a specific set inspiratory time sT_{I}. Likewise, the patient's 12 set tidal volume sV_{T} may also be set directly, or it may be determined by adjusting the patient's 12 inspiratory pressure (P_{INSP}) in, for example, pressure control ventilation (PCV). Adding the patient's 12 set expiratory time sT_{E} to the patient's 12 set inspiratory time sT_{I} results in a breath time that, when divided from 60 seconds, produces the patient's 12 respiratory rate f. Accordingly, the patient's 12 set expiration time sT_{E}, set inspiration time sT_{I}, and respiratory rate f may not be whole numbers.

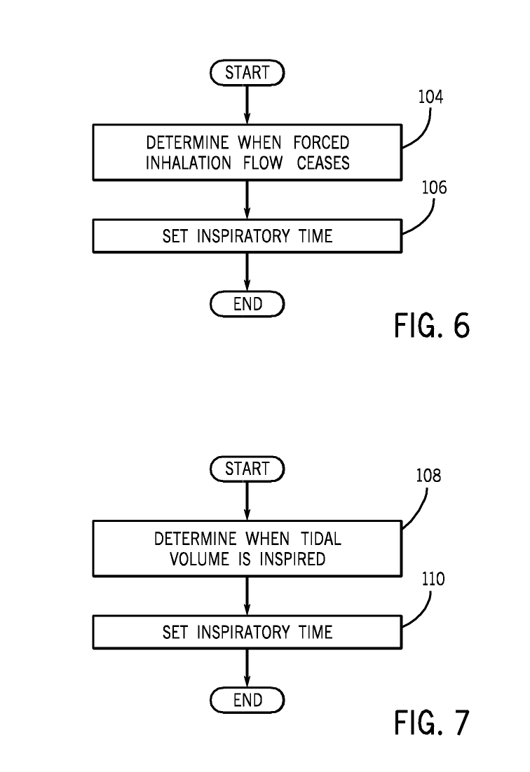

Referring now to FIG. 6, a flowchart depicts a simplified arrangement for setting the patient's 12 set inspiratory time sT_{I} based on when the patient's 12 forced inhalation flow ceases, or again effectively decreases to an insignificant level during a pressure controlled mechanical ventilation delivery mode or the like. More specifically, a method begins in a step 104, during which the patient's 12 forced inhalation flow cessation is determined, or at least effectively decreased to an insignificant amount. Preferably, the patient's 12 effective forced inhalation flow cessation is determined using the patient's 12 airway flow waveform, particularly when the first derivative thereof approaches zero, as is well-known in the art. Alternatively, other arrangements are also well-known in the art and can also be used to determine when the patient's 12 effective forced inhalation flow ceases.

Thereafter, the patient's 12 effective cessation of forced inhalation flow can be used to set the patient's 12 set inspiratory time sT_{I} on the ventilator 16. More specifically, the patient's 12 set inspiratory time sT_{I} can be set based on the patient's 12 effective cessation of forced inhalation flow, and, for example, set equal or substantively equal to when the patient's 12 effective forced inhalation flow ceases, as shown in a step 106 in FIG. 6, after which the method ends.

Referring now to FIG. 7, a flowchart depicts a simplified arrangement for setting the patient's 12 set inspiratory time sT_{I} based on when the patient's 12 tidal volume V_{T} is inspired, particularly during pressure controlled mechanical ventilation. More specifically, a method begins in a step 108, during which inspiration of the patient's 12 tidal volume V_{T} is determined. Preferably, the patient's 12 inspiration of tidal volume V_{T} is determined using a flow sensor. Alternatively, other arrangements are also well-known in the art and can also be used to determine when the patient's 12 tidal volume V_{T} is inspired.

Thereafter, the patient's 12 inspiration of tidal volume V_{T} can be used to set the patient's 12 set inspiratory time sT_{I} on the ventilator 16. More specifically, the patient's 12 set inspiratory time sT_{I} can be set based on the patient's 12 inspiration of tidal volume V_{T}, and, for example, set equal or substantively equal to when the patient's 12 tidal volume V_{T} is inspired, as shown in a step 110 in FIG. 7, after which the method ends.

As previously indicated,

$$f = \frac{60}{{sT}_{I} + {sT}_{E}}$$

$$I \text{:} E = \frac{{sT}_{I}}{{sT}_{E}}$$

whereby knowing the patient's 12 respiratory rate f and I:E ratio allows determining the patient's 12 set inspiratory time sT_{I} and set expiratory time sT_{E}, while knowing the patient's 12 set inspiratory time sT_{I} and set expiratory time sT_{E} conversely allows determining the patient's 12 respiratory rate f and I:E ratio. Preferably, the clinician and/or the ventilator sets the patient's 12 respiratory rate f and set inspiratory time sT_{I}, for which the patient's 12 set expiratory time sT_{E} and I:E ratio can then be determined using the above equations.

While various mandatory mechanical ventilation modes can be used with the inventive techniques, volume guaranteed pressure control ventilation (i.e., PCV-VG), in particular, will be further described below as a representative example, as it has a decelerating flow profile based on the patient's forced inhalation in response to the ventilator delivered inspiratory pressure, and the set tidal volume sV_{T} is guaranteed by the ventilator on a breath-to-breath basis. However, the inventive arrangements are also equally applicable to other pressure control ventilation (PCV) modes. In any event, several of the primary control settings on a typical ventilator 16 include controls for one or more of the following: set expiratory time sT_{E}, set inspiratory time sT_{I}, set tidal volumes sV_{T}, and/or fraction of inspired oxygen F_{I}O₂.

Now then, according to the patient's 12 physiological measurements in a steady state condition:

VĊO₂=F_{ET}CO₂*MV_{A}

wherein VĊO₂ is the volume of C0₂ per minute exhaled by the patient 12 and MV is the minute volume, which is a total volume exhaled per minute by the patient 12. As used in these expressions, a subscripted A indicates “alveolar,” which is a part of the patient's 12 lungs 30 that participate in gas exchanges with the patient's 12 blood, in contrast to deadspace (V_{D}), such as the patient's 12 airway.

In this steady state condition and over a short duration, the patient's 12 blood reservoir is such that VĊO₂ is a constant (blood reservoir effects will be elaborated upon below), and, in accordance with this equation, as MV_{A} increases, the patient's 12 end tidal carbon dioxide F_{ET}CO₂ decreases for a constant VĊO₂. Accordingly, substituting MV_{A}=V_{A}* f yields the following:

$$V \dot{C} {O}_{2} = {F}_{ET} {CO}_{2} * {V}_{A} * f = {F}_{ET} {CO}_{2} * {V}_{A} * \frac{60}{{dT}_{I } + {dT}_{E}}$$

$${V}_{T} = {V}_{A} + {V}_{D}$$

Accordingly, the same VĊO₂ can be achieved by increasing the patient's 12 V_{A} and/or decreasing the patient's 12 respiratory rate f. Decreasing the patient's 12 respiratory rate f has the same effect as increasing the patient's 12 delivered expiratory time dT_{E} on the ventilator 16. In fact, numerous respiratory rate f and delivered expiratory time dT_{E} combinations can result in equivalent or nearly equivalent VĊO₂ production. Accordingly, an optional combination is desired.

As previously described, the patient's 12 forced inhalation time T_{INH} measures the time period when the patient's 12 forced inspiratory gas flow ceases during pressure controlled mechanical ventilation—i.e., the patient's 12 forced inhalation time T_{INH} comprises the duration of gas flow during the patient's 12 delivered inspiratory time dT_{I}. A cessation of flow indicates that the patient's 12 lungs 30 are at their end-inspired lung volume (EILV), subtended by the end-inspired airway pressure. Continued gas exchange beyond EILV could become less efficient, largely as a result of the completion of inspired volume of gases in the patient's 12 lungs 30, and the gases would likely have mixed with the gases already in the patient's 12 lungs 30 since the last exhaled breath.

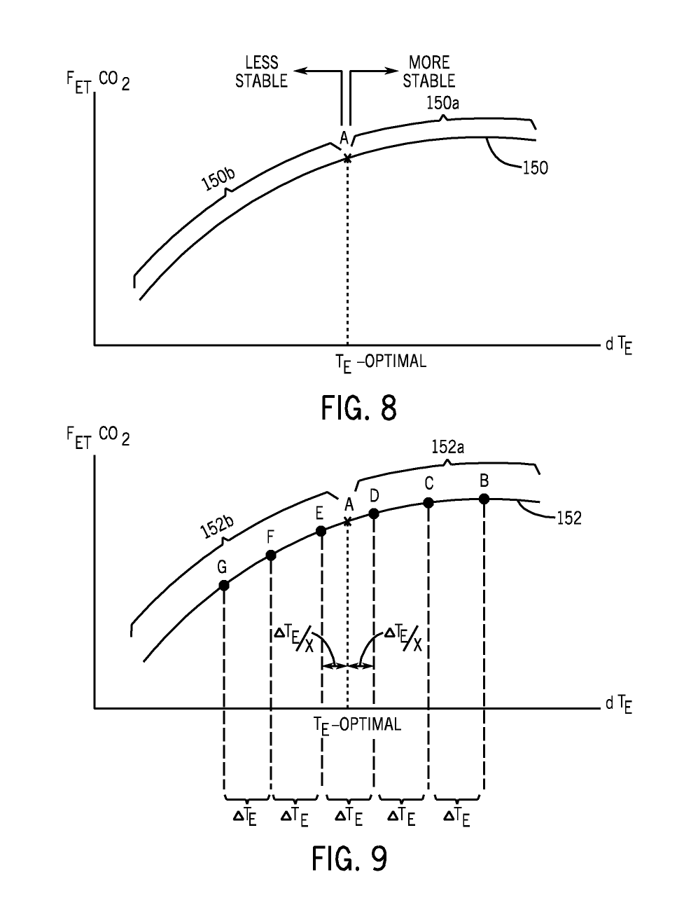

Referring now to FIG. 8, the clinician can also increase or decrease the patient's 12 set expiratory time sT_{E} on the ventilator 16 until the patient's 12 resulting end tidal carbon dioxide F_{ET}CO₂ is or becomes stable to changes in the patient's 12 delivered expiratory time dT_{E}. More specifically, this will identify the patient's 12 optimal expiratory time T_{E-OPTIMAL}. Preferably, the clinician and/or ventilator 16 will be able to determine this optimal expiratory time T_{E-OPTIMAL} within a few breaths of the patient 12 for any given inspiratory cycle. For example, when a stable end tidal carbon dioxide F_{ET}CO₂ is reached, then preferred equilibration of carbon dioxide CO₂ during a given delivered expiratory time dT_{E} can be achieved, as little or no more carbon dioxide CO₂ can be effectively extracted from the patient's 12 blood by further increasing the patient's 12 delivered expiratory time dT_{E}. Accordingly, the patient's 12 optimal expiratory time T_{E-OPTIMAL} can then be ascertained and/or set.

More specifically, the patient's 12 end tidal carbon dioxide F_{ET}CO₂ can be considered stable or more stable at or after a point A on a dT_{E} response curve 150 in the figure (e.g., see a first portion 150a of the dT_{E} Response Curve 150) and non-stable or less stable or instable at or before that point A (e.g., see a second portion 150b of the dT_{E} Response Curve 150). Accordingly, the point A on the dT_{E} Response Curve 150 can be used to determine the patient's 12 optimal expiratory time T_{E-OPTIMAL}, as indicated in the figure.

Physiologically, when the patient's 12 end tidal carbon dioxide F_{ET}CO₂ is equal to the patient's 12 capillary carbon dioxide FcCO₂, diffusion stops and carbon dioxide CO₂ extraction from the patient's 12 blood ceases. Ideally, the patient's 12 optimal expiratory time T_{E-OPTIMAL} is set where this diffusion becomes ineffective or stops. Otherwise, a smaller delivered expiratory time dT_{E} could suggest that additional carbon dioxide CO₂ could be effectively removed from the patient's 12 blood, while a larger delivered expiratory time dT_{E} could suggest that no additional carbon dioxide CO₂ could be effectively removed from the patient's 12 blood.

Preferably, finding the patient's 12 stable end tidal carbon dioxide F_{ET}CO₂ occurs without interference from the patient's 12 blood chemistry sequalae. A preferred technique for finding the patient's 12 stable end tidal carbon dioxide F_{ET}CO₂ can increase or decrease the patient's 12 expiratory time dT_{E}, which may minimally disrupt the patient's 12 blood reservoir of carbon dioxide CO₂. Changes in the patient's 12 delivered expiratory time dT_{E} will affect how the patient's 12 blood buffers the patient's 12 carbon dioxide CO₂, and if that blood circulates back to the patient's 12 lungs 30 before the patient's 12 set expiratory time sT_{E} is optimized, then the patient's 12 end tidal carbon dioxide F_{ET}CO₂ will be different for a given expiratory time dT_{E}. At that point, optimizing the patient's 12 set expiratory time sT_{E} may become a dynamic process. In any event, the time available to find the patient's 12 optimal expiratory time T_{E-OPTIMAL} may be approximately one (1) minute for an average adult patient 12.

One way to decrease the likelihood of interference from the patient's 12 blood chemistry sequalae is to change the patient's 12 delivered expiratory time dT_{E} for two (2) or more expirations, and then use the patient's 12 resulting end tidal carbon dioxide F_{ET}CO₂ to extrapolate using an apriori function, such as an exponential function, by techniques known in the art.

For example, if the patient's 12 first end tidal carbon dioxide F_{ET}CO₂ was originally determined at a point B on a dT_{E} response curve 152 in the figure, and then at a point C, and then at a point D, and then at a point E, and then at a point F, and then at a point G, and then so on, then the data points (e.g., points B-G) could be collected and a best fit dT_{E} response curve 152 obtained; extrapolating as needed. Preferably, the dT_{E} response curve 152 is piecewise continuous. For example, a first portion 152a of the dT_{E} response curve 152 may comprise a stable horizontal or substantially horizontal portion (e.g., points B-D) while a second portion 152b thereof may comprise a polynomial portion (e.g., points E-G). Where this first portion 152a and second portion 152b of the dT_{E} response curve 152 intersect (e.g., see point A on the dT_{E} response curve 152) can be used to determine the patient's 12 optimal expiratory time T_{E-OPTIMAL}, as indicated in the figure.

For example, referring now to FIG. 9, an arrangement to identify the patient's 12 optimal expiratory time T_{E-OPTIMAL} based on an iterative process will be described. More specifically, one preferred arrangement for determining an optimal expiratory time T_{E-OPTIMAL} collects F_{ET}CO₂ data in equal or substantially equal expiratory time increments ATE. For example, if the patient's 12 first end tidal carbon dioxide F_{ET}CO₂ was originally determined to be within the first portion 152a of the dT_{E} response curve 152 (e.g., see points B-D), then the clinician and/or ventilator 16 could decrease the patient's 12 delivered expiratory times dT_{E} until the patient's 12 end tidal carbon dioxide F_{ET}CO₂ readings were within the second portion 152b of the dT_{E} response curve 152 (e.g., see points E-G).

For example, if the patient's 12 end tidal carbon dioxide F_{ET}CO₂ was originally determined to be at point C on the dTE response curve 152 (i.e., within the first portion 152a of the dT_{E} Response Curve 152), then the patient's 12 delivered expiratory time dT_{E} could be decreased until the patient's 12 next end tidal carbon dioxide F_{ET}CO₂ was determined to be at point D on the dT_{E} response curve 152, at which point the patient's 12 end tidal carbon dioxide F_{ET}CO₂ would still be determined to be within the first portion 152a of the dT_{E} response curve 152. Accordingly, the patient's 12 delivered inspiratory time dT_{I} could be decreased again until the patient's 12 next end tidal carbon dioxide F_{ET}CO₂ was determined to be at point E on the dT_{E} response curve 152, at which point the patient's 12 end tidal carbon dioxide F_{ET}CO₂ would now be determined to be within the second portion 152b of the dT_{E} response curve 152 (i.e., the patient's 12 end tidal carbon dioxide F_{ET}CO₂ would have dropped and thus not be at the patient's 12 optimal expiratory time T_{E-OPTIMAL}). Accordingly, a smaller delivered expiratory time increment ATE/x could be made to determine when the patient's 12 end tidal carbon dioxide F_{ET}CO₂ was as at point A on the dT_{E} response curve 152—i.e., at the intersection of the first portion 152a of the dT_{E} response curve 152 and the second portion 152b of the dT_{I} response curve 152. In this iterative fashion, successively smaller delivered time increments and/or decrements ΔT_{E} are made to determine the patient's 12 optimal expiratory time T_{E-OPTIMAL}, as indicated in the figure.

In like fashion, if the patient's 12 end tidal carbon dioxide F_{ET}CO₂ was originally determined to be at point F on the dT_{E} response curve 152 (i.e., within the second portion 152b of the dT_{E} response curve 152), then the patient's 12 delivered expiratory time dT_{E} could be increased until the patient's 12 next end tidal carbon dioxide F_{ET}CO₂ was determined to be at point E on the dT_{E} response curve 152, at which point the patient's 12 end tidal carbon dioxide F_{ET}CO₂ would still be determined to be within the second portion 152b of the dT_{E} response curve 152. Accordingly, the patient's 12 delivered expiratory time dT_{E} could be increased again until the patient's 12 next end tidal carbon dioxide F_{ET}CO₂ was determined to be at point D on the dT_{E} response curve 152, at which point the patient's 12 end tidal carbon dioxide F_{ET}CO₂ would now be determined to be within the first portion 152a of the dT_{E} response curve 152 (i.e., the patient's 12 end tidal carbon dioxide F_{ET}CO₂ would not have increased and thus not be at the patient's 12 optimal expiratory time T_{E-OPTIMAL}). Accordingly, a smaller delivered expiratory time decrement ΔT_{E}/x could be made to determine when the patient's 12 end tidal carbon dioxide F_{ET}CO₂ was as at point A on the dT_{E} response curve 152—i.e., at the intersection of the first portion 152a of the dT_{E} response curve 152 and the second portion 152b of the dT_{E} response curve 152. In this iterative fashion, successively smaller delivered time increments and/or decrements ΔT_{E} are again made to determine the patient's 12 optimal expiratory time T_{E-OPTIMAL}, as indicated in the figure.

In addition, once the patient's 12 optimal expiratory time T_{E-OPTIMAL} is determined, it is realized this may be dynamic, by which the above arrangements can be repeated, as needed and/or desired.

Now then, a lower bound on the patient's 12 set expiratory time sT_{E} should be directly related to the minimal time required for the patient 12 to exhale the delivered tidal volume dV_{T}.

A lower bound for the patient's 12 set and delivered tidal volume sV_{T}, dV_{T} should exceed V_{D}, preferably within a predetermined and/or clinician-selected safety margin. Preferably, a re-arrangement of the Enghoff-Bohr equation can be used to find V_{D} or the following variation:

$${V}_{D} = {V}_{T} - {V}_{A} = {V}_{T} - \frac{{VCO}_{2}}{{F}_{ET} {CO}_{2}}$$

After the patient's 12 end tidal carbon dioxide F_{ET}CO₂ is determined, then the patient's 12 set tidal volume sV_{T} can be set accordingly, but it may not yet be set at an optimal value. Often, the clinician and/or ventilator 16 will attempt to determine this desired value. For example, the clinician may consider the desired value as the patient's 12 pre-induction end tidal carbon dioxide F_{ET}CO₂. The clinician can then adjust the patient's 12 set tidal volume sV_{T} until the desired end tidal carbon dioxide F_{ET}CO₂ is achieved. Alternatively, or in conjunction therewith, a predetermined methodology can also be used to adjust the patient's 12 delivered tidal volume dV_{T} until the desired end tidal carbon dioxide F_{ET}CO₂ is achieved. For example, such a methodology may use a linear method to achieve a desired end tidal carbon dioxide F_{ET}CO₂.

Preferably, the clinician can be presented with a dialog box on the monitor 38, for example (see FIG. 1), indicating the current and/or updated optimal ventilator 16 settings to be accepted or rejected. Preferably, the settings can be presented to the clinician in the dialog box for acceptance or rejection, who can then accept them, reject them, and/or alter them before accepting them. Alternatively, the settings can also be automatically accepted, without employing such a dialog box.

As previously indicated, different techniques can also be used to search for optimal settings for the ventilator 16. If desired, the delivered values can also be periodically altered to assess whether, for example, the settings are still optimal. Preferably, these alterations can follow one or more of the methodologies outlined above, and they can be determined based on a predetermined and/or clinician-selected time interval, on demand by the physiological, and/or determined by other control parameters, based, for example, on clinical events, such as changes in the patient's 12 end tidal carbon dioxide F_{ET}CO₂, or on clinical events such as changes in drug dosages, repositioning the patient, surgical events and the like. For example, the patient's 12 delivered expiratory time dT_{E} can vary about its current value set expiratory time sT_{E} and the resulting end tidal carbon dioxide F_{ET}CO₂ can be compared to the current end tidal carbon dioxide F_{ET}CO₂ to assess the optimality of the current settings. If, for example, a larger delivered expiratory time dT_{E} leads to a larger end tidal carbon dioxide F_{ET}CO₂, then the current set expiratory time sT_{E} could be too small.

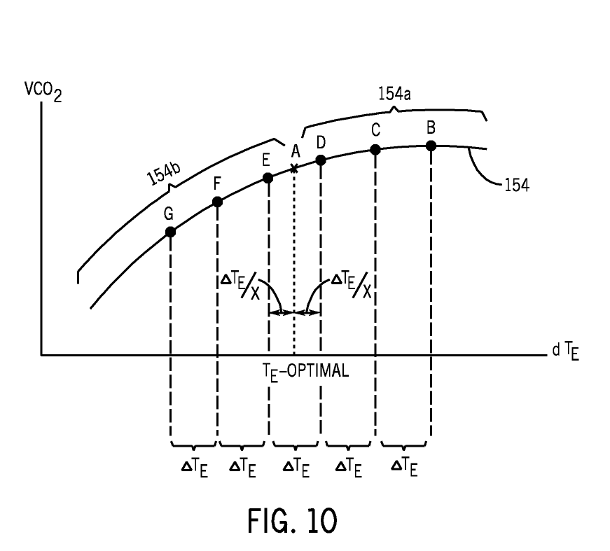

In an alternative embodiment, the dT_{E} response curve 154 could be expressed in terms of VCO₂ instead of F_{ET}CO₂, as shown in FIG. 10. The morphology of the response curve 154 will be similar to that as shown in FIG. 9. Without loss of generality, the above techniques can be used to find T_{E-OPTIMAL} utilizing VCO₂ as opposed to F_{ET}CO₂. The VCO₂ is equal to the inner product over one breath between a volume curve and a CO₂ curve. The flow and CO₂ curves should be synchronized in time.

One representative summary of potential inputs to, and outputs from, such a methodology is depicted below:

| | |
|---|---|
| Clinician Inputs | The patient's 12 age, weight, height, gender, location, |
| | and/or desired F_{ET}CO₂, etc. |
| Measured Inputs | End tidal carbon dioxide F_{ET}CO₂, flow wave data, etc. |
| Outputs | The patient's 12 set inspiratory time sT_{I}, expiratory |
| | time set sT_{E}, and/or set tidal volume sV_{T} |

In addition, by more closely aligning the patient's 12 set inspiratory time sT_{I} and the patient's 12 forced inhalation time T_{INH} during mandatory mechanical ventilation, mean alveolar ventilation increases. In addition, there is additional optimal carbon dioxide CO₂ removal, improved oxygenation, and/or more anesthesia agent equilibration, whereby ventilated gas exchanges become more efficient with respect to use of lower set tidal volume sV_{T} compared to conventional settings. Minute ventilations and respiratory resistance can be reduced, and reducing volumes can decrease the patient's 12 airway pressure P_{aw} thereby reducing the risk of inadvertently over distending the lung.

In addition, the inventive arrangements facilitate ventilation for patients 12 with acute respiratory distress syndrome, and they can be used to improve usability during both single and double lung ventilations, as well transitions therebetween.

As a result of the foregoing, several of the inventive arrangements set the patient's 12 set inspiratory time sT_{I} equal to the time period between when the ventilator 16 permits the patient 12 to inhale and when the patient's 12 inspiratory flow ceases—i.e., the patient's 12 forced inhalation time T_{INH}. This facilitates the patient's 12 breathing by ensuring that ventilated airflows are appropriate for that patient 12 at that time in the treatment. In addition, methods of setting optimal patient expired time T_{E-OPTIMAL} and desired tidal volume V_{T} are presented.

It should be readily apparent that this specification describes illustrative, exemplary, representative, and non-limiting embodiments of the inventive arrangements. Accordingly, the scope of the inventive arrangements are not limited to any of these embodiments. Rather, various details and features of the embodiments were disclosed as required. Thus, many changes and modifications—as readily apparent to those skilled in these arts—are within the scope of the inventive arrangements without departing from the spirit hereof, and the inventive arrangements are inclusive thereof. Accordingly, to apprise the public of the scope and spirit of the inventive arrangements, the following claims are made:

What is claimed is:

1. A method of setting expiratory time in controlled mechanical ventilation, comprising:
varying a subject's expiratory times;
determining end tidal gas concentrations associated with said expiratory times;
establishing a stable condition of said gas concentrations; and
determining an optimal expiratory time based on a deviation from said stable condition.

2. The method of claim 1, wherein said gas concentrations include one or more gases exhaled by said subject.

3. The method of claim 2, wherein at least one of said gases includes carbon dioxide.

4. The method of claim 2, wherein at least one of said gases includes oxygen.

5. The method of claim 2, wherein at least one of said gases includes nitrous oxide.

6. The method of claim 2, wherein at least one of said gases includes inhaled anesthetic agent.

7. The method of claim 1, further comprising:
decreasing said subject's expiratory times to determine said optimal expiratory time.

8. The method of claim 7, wherein said optimal expiratory time corresponds to a smallest expiratory time associated with said deviation.

9. The method of claim 1, further comprising:
increasing said subject's expiratory times to determine said optimal expiratory time.

10. The method of claim 9, wherein said optimal expiratory time corresponds to a smallest expiratory time associated with said deviation.

11. The method of claim 1, further comprising:
displaying at least one or more of the following on a monitor:
said expiratory times; or
said gas concentrations.

12. The method of claim 1, wherein said expiratory times vary by predetermined amounts.

13. The method of claim 12, wherein said predetermined amounts vary over time.

14. The method of claim 1, further comprising setting additional expiratory times based on said optimal expiratory time.

15. A device for use in controlled mechanical ventilation, comprising:
means for varying a subject's expiratory times;
means for determining end tidal gas concentrations associated with said expiratory times;
means for establishing a stable condition of said gas concentrations; and
means for determining an optimal expiratory time based on a deviation from said stable condition.
